(19) United States
(12) Patent Application Publication
(10) Pub. No.: US 2005/0220777 A1
(43) Pub. Date: Oct. 6, 2005

(54) Selection of lactic acid bacteria enhancing the immune response against Streptococcus pneumoniae

(76) Inventors: Stefan Roos, Uppsala (SE); Torun Wall, Uppsala (SE); Hans Jonsson, Balinge (SE); Bo Mollstam, Lerum (SE)

Correspondence Address: Lynn E. Barber, Post Office Box 16528, Fort Worth, TX, 76162, US

(21) Appl. No.: 10/991,891

(22) Filed: Nov. 18, 2004

Related U.S. Application Data
(60) Provisional application No. 60/523,678, filed on Nov. 21, 2003.

(51) Int. Cl. A61K 45/00; C12N 1/20

(52) U.S. Cl. 424/93.45; 435/252.9

(57) ABSTRACT

A method of selection of lactic acid bacteria for providing a method of protecting humans against Streptococcus pneumoniae infections by selecting a lactic acid bacterial strain (LAB) naturally carrying genes coding for proteins similar to the PspC, choline binding protein of S. pneumoniae so that the selected LAB can be used in various products for oral and nasal administration to humans as immune enhancers against S. pneumoniae, and products containing specifically selected LAB that naturally carry genes coding for proteins similar to PcpC binding protein of S. pneumoniae as oral and nasal immune enhancers for humans.

### CROSS-REFERENCE TO RELATED APPLICATIONS

This application claims priority from U.S. Provisional Application, Ser. No. 60/523,678, filed Nov. 21, 2003, which is incorporated herein by reference.

### BACKGROUND OF THE INVENTION

1. Field of the Invention

The present invention relates generally to lactic acid bacteria (LAB) to protect against Streptococcus pneumoniae and more specifically it relates to the selection of lactic acid bacteria enhancing the immune response for providing a method of protecting humans against S. pneumoniae infections by selecting a lactic acid bacteria naturally carrying genes coding for proteins similar to the PspC, choline binding protein of S. pneumoniae, so that the selected LAB can be used in various products for oral and nasal administration to humans as immune enhancers against S. pneumoniae.

2. Description of the Related Art

Pneumonia and invasive pneumococcal disease are a major cause of childhood mortality, 90% of which occurs in developing countries. Routine childhood vaccination would be a cost-effective method for preventing this mortality. Pneumococcal polysaccharide (PPS) vaccines have been available since 1977 but are not immunogenic in infants and young children who comprise the largest proportion of those affected by pneumococcal disease. A 7-valent pneumococcal conjugate (PnC) vaccine was recently licensed in the US and in other developed countries. However, these vaccines cover only 40-70% of the serotypes causing invasive disease in developing countries in Asia and Africa. The next generation of PnC vaccines comprising of 9 or 11 serotypes are currently being evaluated in developing countries. While these vaccines would cover the majority (70-80%) of the invasive serotypes of pneumococcus in children world-wide, and initial results show that these are very safe and efficacious vaccines, there are a few unresolved issues related to these vaccines. The main technical problem is the potential for replacement disease with non-vaccine serotypes that may attenuate the overall benefit seen from reduction in disease due to vaccine serotypes. This phenomenon has been documented with otitis media in a trial in Finland, but not with invasive disease. It is currently not known whether this would occur with pneumonia, the primary outcome of interest for developing countries. There are also several other concerns about the conjugate vaccines related to the cost and complexity of manufacture, and problems with registration of newer candidates in the United States. However, it is unclear whether these would be sufficient impediments to the introduction of these vaccines in developing countries. The parallel development of alternate pneumococcal vaccine strategies offers the potential for extending the protective capabilities of conjugate vaccines by providing broader serotype coverage, which may also overcome the problem of serotype replacement, as well as the cover other high risk target groups who may not, be covered by the current conjugate vaccine formulations, e.g. the elderly. A new and potentially faster and cheaper method would be that of the invention, namely to use selected lactic acid bacteria (LAB) naturally carrying genes coding for PspC similar proteins in products for oral or nasal use, to create immunity against S. pneumoniae in humans.

It can be appreciated that vaccines to protect against S. pneumoniae have been in use for years. Typically, vaccine strategies to protect against S. pneumoniae are comprised of prevention of pneumococcal infections by immunization with vaccines which contain capsular polysaccharides from the most common serotypes causing invasive disease. For the future, protein antigens that are conserved across all capsular serotypes that would induce more effective and durable humoral immune responses and could potentially protect against all clinically relevant pneumococcal capsular types are being investigated as components for new generations of improved pneumococcal vaccines.

The main problem with conventional vaccines to protect against S. pneumoniae is that there are more than 90 antigenically distinct serotypes of S. pneumoniae, and there is concern that serotypes not included in the vaccines may become more prevalent in the face of continued use of polysaccharide vaccines. Another problem with conventional vaccines to protect against S. pneumoniae is that certain high-risk groups, such as infants and children, have poor immunological responses to some of the polysaccharides in the vaccine formulations. Another main technical problem is the potential for replacement disease with non-vaccine serotypes that may attenuate the overall benefit seen from reduction in disease due to vaccine serotypes. A problem with the current attempts to create enhanced immunity specifically against the PspC binding protein of S. pneumoniae using other more safe bacteria as “delivery systems” of the antigen, is that it will require complicated development work, production and regulatory approval.

While these prior products may be suitable for the particular purpose which they address, they are not as suitable for providing a method of protecting humans against S. pneumoniae infections as is selecting a lactic acid bacteria (LAB) strain naturally carrying genes coding for proteins similar to the PspC, choline binding protein of S. pneumoniae, so that the selected LAB can be used in various products for oral and nasal administration to humans as immune enhancers against S. pneumoniae.

In these respects, the selection of LAB strains enhancing the immune response according to the present invention substantially departs from the conventional concepts and designs of the prior art, and in so doing provides products primarily developed for the purpose of providing a method of protecting humans against S. pneumoniae infections. In view of the foregoing disadvantages inherent in the known types of vaccines to protect against S. pneumoniae now present in the prior art, the present invention provides new products by selecting a lactic acid bacteria (LAB) naturally carrying genes coding for proteins similar to the PspC, choline binding protein of S. pneumoniae, so that such selected LAB can be used in various products for oral and nasal administration to humans as immune enhancers against. Streptococcus pneumoniae.

A primary object of the present invention is to provide a selection of products with lactic acid bacteria enhancing the immune response against S. pneumoniae that will overcome the shortcomings of the prior art methods and products.

An object of the present invention is to provide a method of protecting humans against S. pneumoniae infections by selecting a lactic acid bacteria (LAB) strain(s) naturally carrying genes coding for proteins similar to the PspC, choline binding protein of S. pneumoniae so that the such selected LAB can be used in various products for oral and nasal administration to humans as immune enhancers against Streptococcus pneumoniae.

Another object is to provide a selection of products with lactic acid bacteria enhancing the immune response that can be used in combination with vaccines, to enhance the immune response against S. pneumoniae in humans.

Another object is to provide a method of selecting one or more lactobacilli (LAB) strain(s) naturally carrying genes coding for proteins similar to the PspC, choline binding protein of S. pneumoniae so that the such selected LAB strain(s) can be used as the immune enhancer of the present invention.

Another object is to provide a selection of LAB strains enhancing the immune response that is GRAS (generally regarded as safe).

Another object is to provide a selection of lactic acid bacteria enhancing the immune response that is relatively easy to select, produce and administer to humans in the risk group, and is therefore more cost effective than presently known methods.

Other objects and advantages of the present invention will become obvious to the reader and it is intended that these objects and advantages are within the scope of the present invention.

### SUMMARY OF THE INVENTION

A method of selection of lactic acid bacteria to protect humans against Streptococcus pneumoniae infections by selecting a lactic acid bacterial strain (LAB) naturally carrying genes coding for proteins similar to the PspC, choline binding protein of S. pneumoniae so that the selected LAB can be used in various products for oral and nasal administration to humans as immune enhancers against S. pneumoniae, and products containing specifically selected LAB that naturally carry genes coding for proteins similar to PcpC binding protein of S. pneumoniae as oral and nasal immune enhancers for humans.

Possible strains to select from are lactic acid bacteria in general, one such example being Lactobacillus reuteri, as seen from the example below. Also strains from Lactobacillus plantarum, Lactobacillus sakei and others have been reported to have similar genes.

### DETAILED DESCRIPTION OF THE INVENTION AND PREFERRED EMBODIMENTS THEREOF

The general purpose of the present invention, which will be described subsequently in greater detail, is to provide new products based on the invention that has many of the advantages of a vaccine to protect against S. pneumoniae mentioned heretofore, and many novel features that result from the described selection of lactic acid bacteria enhancing the immune response against S. pneumoniae. To attain this, the present invention generally comprises products containing specifically selected LAB that naturally carry genes coding for proteins similar to PcpC binding protein of S. pneumoniae as oral and nasal immune enhancers for humans and a method for selecting such LAB strains.

There has thus been outlined, rather broadly, the more important features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application and is capable of other embodiments and of being practiced and carried out in various ways. Also, it is to be understood that the phraseology and terminology employed herein are for the purpose of the description and should not be regarded as limiting.

The invention includes products containing selected LAB strain(s) that naturally carry genes coding for PspC similar proteins. The products are made containing selected lactic acid bacteria (LAB) that naturally carry genes coding for proteins similar to PspC, the choline binding protein of S. pneumoniae so that the such selected LAB strain(s) used in various products for humans will act as immune enhancers against infection caused by Streptococcus pneumoniae. The products are typical and new formulations of LAB for oral administration and also old and new formulations for nasal use. Examples of products for oral use are: yogurts and other fermented food products, drinks and milk products containing the selected LAB with or without fermentation, clinical nutrition, powders, capsules, tablets, caps, drinking straws etc.

The invention herein relates to selecting S. pneumoniae immune enhancing LAB strain(s). The idea of the selection method is to find lactic acid bacteria carrying genes coding for proteins similar to PspC, the choline binding protein of S. pneumoniae so they can be used as described in the products of the present invention. The selection can be done using several different tools such as analysis of whole or parts of the genome of different LAB strains, for example, by construction and screening of a phage display library by insertion of randomly fragmented chromosomal DNA isolated from the LAB strains into a vector for extra-cellular proteins. After affinity selection, the LAB inserts can be sequenced and analyzed using standard bioinformatic tools. After such genes have been found and the LAB has been selected by the use of the method, the LAB strains are evaluated to determine if the PspC-similar protein is expressed under the conditions that will be used when the products are applied. Other ways to determine if a particular LAB strain expresses proteins similar to the PspC can be by bioinformatic analysis of various complete or partly available genome of LAB. Also can antibodies raised against PspC be used.

The invention of using LAB strains naturally containing genes coding for PspC similar proteins, and expressing such proteins under the conditions of use of the products of the invention is built upon a method described herein to find such LAB, and then formulate the LAB into products for oral or nasal use in humans.

As to a further discussion of the manner of usage and operation of the present invention, the same should be apparent from the above description.

### EXAMPLE 1

### Selection of a Suitable Strain

Bacterial strains, growth conditions, helper phase and phagemid vector. Escherichia coli strain TG1 (Sambrook, J., Fritsch, E. F. & Maniatis, T. (1989). Molecular Cloning: a Laboratory Manual, 2nd edn. Cold Spring Harbor, N.Y.: Cold Spring Harbor Laboratory) was utilized to construct the phage display library and to produce phage stocks. The disclosure of this publication and all other publications referred to herein is incorporated herein by reference. E. coli cells were grown in Luria-Bertani broth (LB) (Sambrook et al., 1989) and E. coli transformants in LB with 50 μg ampicillin ml⁻¹ (LB/Amp) at 37° C. with shaking. Solid medium for growth of E. coli transformants was obtained by addition of 1•5% (w/v) agar to LB/Amp. Lactobacillus reuteri DSM 20016T was grown in Man-Rogosa-Sharpe (MRS) broth (Oxoid) at 37° C. Phage R408 (Promega) was used as helper phage and pG3DSS (Rosander, A., Bjerketorp, J., Frykberg, L. & Jacobsson, K. (2002). Phage display as a novel screening method to identify extracellular proteins. J Microbiol Methods 51, 43-55.)

Isolation of chromosomal DNA from L. reuteri DSM 20016T. For construction of the library, chromosomal DNA was isolated from L. reuteri. Ten milliliters of an overnight culture of the bacterium was added to 300 ml MRS broth and grown to an OD600 of approximately 1.5. Cells were harvested by centrifugation at 4000 g for 15 min, washed in 50 ml 10 mM Tris/HCl (pH 8.0)/0.3 M sucrose (T-sucrose) and resuspended in 50 ml T-sucrose supplied with 20 mg lysozyme ml⁻¹ and 160 U mutanolysin ml⁻¹ (Sigma). The suspension was incubated for 2 h at 37° C. with slow shaking; the cells were then pelleted by centrifugation and resuspended in 9 ml 50 mM Tris/HCl (pH 8.0)/5 mM EDTA (5×TE). After addition of 1 ml 10% (w/v) SDS, the suspension was carefully mixed and incubated at 65 ° C. for 15 min. Next, RNase was added to a final concentration of 100 μg ml⁻¹ and the incubation was continued for 30 min at 37° C. Proteinase K was added to a final concentration of 200 μg·ml⁻¹ and the suspension was incubated at 55° C. for 30 min. Finally, the DNA was precipitated by addition of 1 ml 3 M sodium acetate (pH 5.2) and 20 ml 95% ethanol, washed in 70% ethanol, and dissolved in 5 ml 1×TE. After repeated phenol and chloroform extractions, the DNA was precipitated again, washed in 70% ethanol and dissolved in an appropriate volume of 1×TE.

For DNA sequencing with chromosomal DNA as template, L. reuteri DNA was isolated with Qiagen Genomic-tips 500/G according to the manufacturer's instructions, with one modification. For lysis of the bacteria, in addition to lysozyme (final concentration 10 mg ml⁻¹), mutanolysin was added to the cell suspension to a final concentration of 80 U ml⁻¹. After incubation for 30 min at 37° C. with gentle shaking, Proteinase K (final concentration 0.9 mg ml⁻¹) was added and the incubation was continued for 30 min. Construction of the library. The library was constructed as described by Jacobsson, K., Rosander, A., Bjerketorp, J. & Frykberg, L. (2003). (Shotgun phage display—selection for bacterial receptins or other exported proteins. Biol Proced Online 5, 123-135.) with minor modifications. Chromosomal DNA (400 μl, 150 μg ml⁻¹) was sonicated for 10 s at maximum power with a microprobe (Soniprep 150, MSE). The fragments obtained varied in size between 0•5 and 2 kb, with the majority between 0.7 and 1.3 kb. Blunt ends were achieved by treatment with T4 DNA polymerase. The phagemid vector pG3DSS (Rosander et al., 2002, J. Microbiol. Methods, 51:43-55) was digested with the restriction enzyme SnaBI and dephosporylated with calf intestine alkaline phosphatase. The DNA manipulations were performed according to standard methods (Sambrook et al., 1989).

Five tubes of Ready-To-Go T4 DNA Ligase (Amersham Pharmacia Biotech) were utilized to ligate approximately 1 μg (0.2 μg per tube) of L. reuteri fragments with approximately 3 μg (0.6 μg per tube) of the vector pG3DSS. After phenol and chloroform extraction, the ligated DNA was ethanol-precipitated, washed with 70% ethanol and dissolved in 10 μl H₂O. The ligation mix was transformed into E. coli TG1 by electroporation (2.5 kV, 25 μF, 400) in 2-mm-gap cuvettes. The transformed cells were transferred to 100 ml LB and incubated at 37° C. for 1 h with gentle shaking. Then, a 2 ml aliquot was taken from the culture to determine the number of transformants by plating on LB/Amp agar. After addition of ampicillin to a final concentration of 50 μg ml⁻¹, the remaining cell culture was grown overnight at 37° C. with vigorous shaking.

An 8 ml aliquot of the overnight culture was infected with helper phage R408 (m.o.i. 5), mixed with 50 ml soft agar (LB broth with 0•5%, w/v, agarose) and poured onto 10 LB/Amp agar plates. The plates were incubated overnight at 37° C., then the phages were extracted from the soft agar. Finally, the titer of the phage library was determined by infection of E. coli TG1.

Panning procedures. In order to isolate phages displaying fusion proteins, the library was panned against anti-E-tag antibodies (Amersham Biosciences). Nunc-immuno modules (MaxiSorp) were coated with 200 μl anti-E-tag antibodies (final concentration 25 μg ml⁻¹), or as a control with 0.1% (w/v) BSA, in 50 mM sodium carbonate (pH 9.7) and the wells were blocked with 400 μl phosphate-buffered saline (Sambrook et al., 1989, Molecular Cloning: a Laboratory Manual)/0.05% Tween 20 (v/v) (PBS-T). After addition of 200 μl of the phage library to the wells, the phagemid particles were allowed to bind to the anti-E-tag antibodies during 4 h incubation with slow agitation at room temperature (22° C.). The wells were rinsed thoroughly with PBS-T (30 times), bound phages were eluted by addition of 200 μl 50 mM sodium citrate/140 mM NaCl (pH 2•1) and the eluate was neutralized with 30 μl 2 M Tris/HCl (pH 8•0). An overnight culture of E. coli TG1 was infected with the eluate and the infected cells were plated on LB/Amp agar. After resuspension in LB, the clones obtained were infected with helper phage R408 (as described under Construction of the library) to produce a phage stock for repanning. To enrich for positive clones (i.e. those displaying fusion proteins), this stock was utilized for a second panning, which was performed as described above. The procedure of panning and repanning was repeated in two separate experiments.

Nucleotide Sequencing of L. reuteri Inserts and of L. reuteri Chromosomal DNA.

After panning, 120 clones from the first panning cycles and 143 clones from the repannings were randomly selected for nucleotide sequencing of the L. reuteri DNA inserts. The phagemid vectors from these clones were isolated with the QIAprep Spin Miniprep Kit (Qiagen). The DNA fragments inserted in the vector were sequenced with the primer G3DelRev (5′-cattgacaggaggttgaggc-3′) or the primer pG3DR2 (5′-gcggttccagtgggtcc-3′), with the DYEnamicET terminator cycle sequencing premix kit (Amersham Biosciences) and an ABI PRISM 377 DNA Sequencer (PerkinElmer Instruments).

Since the L. reuteri fragments encoded the 5′ end of genes and not complete genes, additional sequence information was required for the majority of the genes. Therefore, primers were designed for sequencing the DNA downstream of the inserts using L. reuteri chromosomal DNA as template. The sequencing of chromosomal DNA was performed as described by Heiner et al. (1998) Genome Res. 8: 557-561. Specifically, in each reaction, 16 μl BigDye Terminator mix v3.0 or v3.1 (Applied Biosystems), 32 pmol primer and 2.5-3.1 μg L. reuteri DNA were added to a total volume of 40 μl. The sequencing reaction was performed with an initial denaturation step at 95° C. for 5 min, followed by 90 cycles at 95° C. for 30 s, 55° C. for 20 s and 60° C. for 4 min.

Sequence analysis. The sequences obtained were analysed with Vector NTI software (InforMax). For prediction of signal sequences, the SignalP V2.0 program for Gram-positive bacteria (Nielsen et al., 1997, Protein Eng. 10: 1-6) was utilized. The signal sequences were verified manually to confirm the presence of the N-, C- and H-regions (van Wely et al., 2001, FEMS Microbiol Rev. 25: 437-454). In addition, translation initiation sequences (reviewed by Kozak, 1999, Gene 234: 187-208) were searched for manually. Similarity studies and searches for conserved domains were performed with BLASTP (Altschul et al., 1990, J. Mol. Biol. 215: 403-410) at the National Centre for Biotechnology Information (NCBI) web page (http://www.ncbi.nlm.nih.gov), using an e-value lower than e-4 as a cut-off for notable similarity. In addition, motifs were searched for with InterProScan (http://www.ebi.ac.uk/interpro/scan.html) (Mulder et al., 2003, Nucleic Acids Res. 31: 315-318, transmembrane domains with TMHMM 2.0 (Krogh et al., 2001, J. Mol. Biol. 305: 567-580) (http://www.cbs.dtu.dk/services/TMHMM-2.0/) and the G+C content of the genes was determined with the program Geecee from EMBOSS (Rice et al., 2000, Trends Genet 16: 276-277) at the Pasteur Institute website (http ://bioweb.pasteur. fr/seqanal/interfaces/geecee.html).

Results and Discussion

Construction of a L. reuteri Phage Display Library and Screening for Extracellular and Transmembrane Proteins

A phage display library was constructed by ligation of randomly fragmented DNA from L. reuteri DSM20016T into the vector pG3DSS. This strain was selected because of its adhesive properties (Jonsson et al., 2001, FEMS Microbiol Lett. 204: 19-22). Additionally, it is comparatively easy to transform (Ahrné et al., 1992, Curr. Microbiol. 24: 199-205), which facilitates molecular analysis. The resulting library consisted of 4•6×107 clones, and after infection with the helper phage R408, the titre of phagemid particles in the library was 1×1011 c.fu. ml⁻¹. The size of the L. reuteri genome was determined by PFGE analysis. Cleavage with NotI generated fragments of 655, 358, 247, 192, 145, 96, 87, 57, 49 and 29 kb, which gave a total size of 1915 kb. Cleavage with SgrAI generated fragments of 1124, 573, 229 and 168 kb, which gave a size of 2094 kb. Subsequently, the size of the L. reuteri genome was estimated to be 2•0±0•2 Mb. The size of the fragments utilized for the library construction was on average 1•0 kb. Expression of the inserted fragments in fusion to the coat protein requires a functional Shine-Dalgarno sequence, as well as the correct orientation and fusion to the right reading frame in the vector (Jacobsson et al., 2003, Biol. Proced. Online 5: 123-135). However, the library covered the genome approximately 20 000 times, which ensured that most genes were correctly fused to the coat protein. Therefore, the library was considered sufficient to screen for genes encoding extracellular proteins.

Portions of the library were utilized for affinity panning against anti-E-tag antibodies. The clones obtained in the first panning were used to produce a phage stock for a second panning to increase the number of positive clones. The number of phagemid particles in the eluate after the panning and repanning is presented in Table 1 of Wall et al. 2004, Microbiology 149: 3493-3505-http://mic.sgmjournals.org:80/cgi/content/full/149/12/3493-T1. In the first experiment, there was a 2500-fold increase in eluted phages after the repanning compared to after the first panning. In the second experiment, the number of phages in the eluate had increased 800-fold between the first panning and the repanning. As a control, portions of the library and the phage stocks for repanning were panned against BSA. After the first pannings, the differences in enriched phages between the controls and the affinity selections against anti-E-tag antibodies were small. In contrast, the repannings against anti-E-tag antibodies contained markedly higher numbers of phagemid particles than the BSA controls. This indicated that positive clones were enriched in the eluate after panning against the anti-E-tag antibodies. It is a common feature of the phage display method that the first panning results in small differences from the control because of an inherent background. However, a clear enrichment is usually established after repanning (Bjerketorp et al., 2002, Microbiology 148: 2037-2044; Jacobsson & Frykberg, 1996, Biotechniques 20: 1070-1081). The number of phagemid particles in the eluate after affinity selection was determined as c.f.u. on LB/Amp plates after infection of E. coli TG1. The library was panned against anti-E-tag antibodies for enrichment of clones displaying fusion proteins and against BSA as a control.

Identification and Classification of Genes Encoding Extracellular and Transmembrane Proteins—Identification of Signal Sequences.

After panning, 120 clones from the first pannings and 143 clones from the repannings were selected for sequencing of the L. reuteri DNA fragments inserted in the vector. The sequences obtained were analysed with Vector NTI software, and the SignalP V2.0 program for Gram-positive bacteria (Nielsen et al., 1997, Protein Eng. 10: 1-6) was utilized for prediction of signal sequences. Of the 263 clones, 162 contained ORFs and expressed genes encoding proteins with signal peptides. The presence of N-, C- and H-regions (van Wely et al., 2001, FEMS Microbiol. Rev. 25: 437-454) of the signal peptides was also confirmed manually. Several of the genes were present in more than one clone, and in total 53 different genes were represented.

The majority of the 53 genes encoding extracellular and transmembrane proteins expressed a classical N-terminal signal peptide recognized by the Sec pathway. In addition, some genes appeared to encode lipoproteins, a subclass of secreted proteins. The precursors of lipoproteins contain signal peptides with a conserved motif called lipobox and the peptides are cleaved by SPase II in front of an invariable cysteine residue (reviewed by Tjalsma et al., 2000, Microbiol. Mol. Biol. Rev. 64: 515-547). For Gram-positive bacteria the motif varies and the consensus is continually refined. Based on the analyses of lipoboxes from Gram-positive bacteria (Sutcliffe & Russell, 1995, J. Bacteriol. 177: 1123-1128) and from Bacillus subtilis (Tjalsma et al., 2000, Microbiol. Mol. Biol. Rev. 64: 515-547) in particular, the consensus sequence has been extracted respectively as (LVAI)-(SALIMTGFV)-(AGLS)-C-(SGNATYWQP) with the C at positions 17-29 and (LVFTIMG)-(ASTIVGMLCPFL)-(AGLISVTFP)-C-(GSAITFWKL) with the C at positions 14-32. By comparing the L. reuteri proteins with these sequences and with the proteins to which they have high similarities, six putative lipoproteins were found in this study. One protein possibly involved in specific interaction between the LAB and its host were identified: Lre0019 with similarities to the S. pneumoniae PspC.

A formulation of a product, for example a nasal spray product, using the selected lactic acid bacteria would follow normal methods and procedures in the industry and would be known by someone skilled in the art. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A method for selecting a lactic acid bacterial strain for protecting a person against Streptococcus pneumoniae infection, comprising selecting a strain of lactic acid bacteria carrying genes coding for proteins sufficiently similar to the choline-binding protein of Streptococcus pneumoniae so that administration of said strain of Lactobacillus enhances the immune response of the person to Streptococcus pneumoniae.

2. The method of claim 1, wherein the selection method comprises construction and screening of a phage display library by insertion of randomly fragmented chromosomal DNA isolated from lactic acid bacterial strains into a vector for extracellular proteins.

3. The method of claim 2, further comprising evaluation of expression of the choline-binding protein under conditions that will be used when a product containing the selected strain is provided to the person.

4. A method of enhancing the immune response of a person against Streptococcus pneumoniae, comprising administering to the person cells of the lactic acid bacterial strain selected by the method of claim 1.

5. The method of claim 4, wherein the cells of the lactic acid bacterial strain are administered as a nasal spray.

6. A product containing cells of a lactic acid bacterial strain selected by the method of claim 1.

7. The product of claim 6, further comprising a suitable vaccine against Streptococcus pneumoniae infection.

8. The product of claim 6 formulated for oral use in humans.

9. The product of claim 6 formulated for nasal use in humans.


SEQUENCE LISTING (4 sequences)

SEQ ID NO: 1 - DNA, 20 bp, Escherichia coli
cattgacaggaggttgaggc

SEQ ID NO: 2 - DNA, 17 bp, Escherichia coli
gcggttccagtgggtcc

SEQ ID NO: 3 - protein, 5 aa, Bacillus subtilis
  MISC_FEATURE (1)..(1): x may be l, v, a, or i
XXXCX

SEQ ID NO: 4 - protein, 5 aa, Bacillus subtilis
  MISC_FEATURE (1)..(1): x may be l, v, f, t, i, m, or g
XXXCX